(19) United States
(12) Patent Application Publication
(10) Pub. No.: US 2013/0023657 A1
(43) Pub. Date: Jan. 24, 2013

(54) SYSTEM FOR RADIOPHARMACEUTICAL PREPARATION INVOLVING SOLID AND LIQUID PHASE INTERACTIONS

(71) Applicants: Thomas A. KLAUSING, Powell, OH (US); Jeffery T. STROUP, Upper Arlington, OH (US); Jason T. HOLDRIDGE, Hilliard, OH (US)

(72) Inventors: Thomas A. KLAUSING, Powell, OH (US); Jeffery T. STROUP, Upper Arlington, OH (US); Jason T. HOLDRIDGE, Hilliard, OH (US)

(73) Assignee: CARDINAL HEALTH 414, LLC, Dublin, OH (US)

(21) Appl. No.: 13/550,018

(22) Filed: Jul. 16, 2012

Related U.S. Application Data
(60) Provisional application No. 61/508,349, filed on Jul. 15, 2011.
(60) Provisional application No. 61/508,294, filed on Jul. 15, 2011.

(51) Int. Cl. G01N 21/75; G01N 30/02; C07H 1/00

(52) U.S. Cl. 536/28.2; 422/68.1; 422/82.05

(57) ABSTRACT

A system and method for radiopharmaceutical production involving solid and liquid phase interactions are provided, the system including a module for facilitating solid and liquid phase interactions by performing techniques including high pressure, low pressure, and solid phase extraction. The system includes various modular components, each of which performs steps in the process of preparing radiopharmaceuticals, and one or more radiation detectors monitor the radiation level and path of various products. The modular components may be added to and removed from the system easily to allow for flexibility in the operation of the system. An HPLC module may be included to purify radiopharmaceuticals.

### RELATED APPLICATIONS

This application claims priority from U.S. Patent Application Nos. 61/508,349, filed on Jul. 15, 2011, and titled “System for Radiopharmaceutical Preparation Including High Performance Liquid Chromatography Module,” and U.S. Provisional Application No. 61/508,294, entitled “Systems, Methods, and Devices for Producing, Manufacturing, and Control of Radiopharmaceuticals,” filed on Jul. 15, 2011. The entirety of each of the preceding applications is incorporated by reference herein.

### BACKGROUND OF THE INVENTION

1. Field of the Invention

The present invention relates to a system for radiopharmaceutical preparation involving solid and liquid phase interactions. In particular, the present invention relates to a system having a high performance liquid chromatography (HPLC) module.

2. Background

Nuclear medicine is a branch of medical imaging that uses small amounts of radioactive materials to diagnose or treat a variety of diseases, including many types of cancers, heart disease, and other abnormalities within the body. For example, positive emission tomography (PET) is a type of nuclear medicine imaging in which a radiopharmaceutical that includes a radionuclide tracer is introduced into the body where it eventually accumulates in an organ or area of the body being examined. The radionuclide gives off energy in the form of gamma rays, which are detected by devices, including a PET scanner. In PET, radiopharmaceuticals that incorporate the radionuclide fluorine-18, such as fluorodeoxyglucose (FDG), 3′-deoxy-3′-[¹⁸F]-fluorothymidine (FLT), [¹⁸F]-fluoromisonidazol (F-MISO), (4-[¹⁸F]-fluorobenzoyl)norbiotinamide (FBB), AV-45, AV-133, and PET Perfusion Agents (PPA), are commonly used.

Due to the radioactive nature of radiopharmaceuticals, special consideration must be taken in their preparation, handling, and delivery. Production of fluorine-18 for use in a radiopharmaceutical is often difficult and expensive, requiring specialized equipment, such as a cyclotron. The production of the radioisotope often occurs at a remote facility by a third party, from which the hospital or lab receives patient doses that are ready to inject. Even if the radioisotope happens to be produced on site, final production of the radiopharmaceuticals used in many diagnostic imaging procedures requires manual preparation in a special aseptic environment to ensure a safe injectable product that is free of environmental contaminants. In addition, precise accounting of the radioactive nature of the radionuclide to be used in the radiopharmaceutical for each procedure is required, while taking into account that the bulk radionuclide product continuously decays over time.

Furthermore, during preparation of radiopharmaceuticals, technicians must be shielded from the ionizing radiation of the radionuclide, and the purity of the radiopharmaceutical must be ensured by filtering and/or avoiding contamination through contact with particles in the air, on a surface, and/or when mixing with a diluting liquid, for example. In addition, because of the short half-life of the radionuclide, the efficient scheduling of patients, for example, along with a safe and efficient preparation of the radiopharmaceutical by technicians is critical to avoid wasting the prepared bulk product of the radionuclide.

Shielded containment systems for use in combining cyclotron-produced radionuclides with non-radionuclide components to produce radiopharmaceuticals have been developed. There are, however, many drawbacks of these systems. In particular, typically only one radiopharmaceutical may be produced in a production run. After a run, various radionuclide raw material components and physical system components must be replaced or decontaminated, which can greatly delay the production process and/or make the process much less efficient. Further, many aspects of production of radiopharmaceuticals in such related art systems are not automated and/or may require time-consuming and/or awkwardly controllable hand production steps. In addition, the radioactivity and/or quantities of the raw radionuclide and/or the produced radiopharmaceutical may be inaccurate and/or difficult to determine precisely. Necessary quality control to be performed on the output radiopharmaceutical products may be time-consuming, inaccurate, and/or require high levels of worker input/skill, further hampering production and/or timely delivery of the produced radiopharmaceuticals.

In addition, to carry out a process in which chemical reactions between a variety of reagents are to take place, such as in the production of radiopharmaceuticals, a large and complex setup is sometimes needed to channel liquids, reagents and/or compounds towards a reactor vessel. Channeling various ingredients towards the reactor vessel generally involves the use of tubing, threaded connectors, valving and the like. Moreover, some ingredients or reagents may have a short shelf life and may have to be used very quickly after manufacture or after exposure to the environment, which increases the need for complex reaction vessels.

Further, various techniques involving solid and liquid phase interactions may be used for purifying the components of a mixture during the production of radiopharmaceuticals, which may also involve a complex setup. For example, high performance liquid chromatography (HPLC) is a technique that is used in a wide range of applications to identify, quantify, and purify the individual components of mixtures. As in other types of chromatography, HPLC involves passing a mixture containing an analyte that has been dissolved in a mobile phase through a stationary phase. The stationary phase is typically contained in a column, and the mobile phase passes through the column. The retention time of each of the components of the mixture varies depending on the strength of its interactions with the stationary phase, the ratio/composition of solvent(s) used, and the flow rate of the mobile phase. Accordingly, each of the components of the mixture flows through the stationary phase at different rates that are based on the affinity of each component for the stationary phase. These differing rates provide separation of the analyte from the other components in the mixture. A specific stationary phase material may be selected to separate a particular component in a mixture. As the components flow out of the column, a detector determines the retention time for the analyte.

HPLC uses a pump to provide high pressure to move the mobile phase and analyte through the column. This allows for better separation of components using columns of shorter length when compared to typical chromatographic techniques, which rely on the pressure from gravity to move components through a column.

HPLC techniques may be used to purify a radiopharmaceutical mixture. The size and complexity of the components used in commercial HPLC units, however, make it impractical to incorporate the HPLC functionality in conventional systems for synthesizing radiopharmaceuticals discussed above. Accordingly, there is a need in the art for systems and methods that incorporate solid and liquid phase interactions for purifying radiopharmaceuticals mixtures and that reduce or eliminate the need for excessive connections, tubing, and the like.

Cadmium Zinc Telluride (CZT) is an alloy of cadmium telluride and zinc telluride that is a direct bandgap semiconductor and that can be used in a variety of applications including radiation detectors, photorefractive gratings, electro-optic modulators, solar cells, and terahertz generation and detection. Radiation detectors using CZT can operate in direct-conversion (or photoconductive) mode at room temperature, and provide the advantages of a high sensitivity for x-rays and gamma-rays because of the high atomic numbers of Cd and Te and better energy resolution than scintillator detectors. CZT can be formed into different shapes for different radiation-detecting applications, and a variety of electrode geometries, such as coplanar grids, have been developed to provide unipolar (electron-only) operation, thereby improving energy resolution.

### SUMMARY

Various aspects of the current invention relate to a system for radiopharmaceutical preparation involving solid and liquid phase interactions. The system may include a module for facilitating solid and liquid phase interactions by performing techniques including, but not limited to, high pressure, low pressure, and solid phase extraction. For example, the system may include an HPLC module. The system may include various modular components, each of which performs steps in the process of preparing radiopharmaceuticals. The modules may be added to and removed from the system easily to allow for flexibility in the operation of the system. According to an aspect of the invention, an HPLC module may be included to purify radiopharmaceuticals.

According to various aspects of the current invention, CZT detectors may also be used to provide detection of radioactive material being provided to an HPLC column, as well as coming out of the HPLC column. For example, CZT detection may be used to determine whether a sample component coming out of an HPLC column is a synthesis material and should be routed to a synthesis module, or whether the sample component should be routed to a waste disposal facility. CZT detection may also be used to determine whether a sample has a level of radiation that is higher than a given threshold, and as such determine, e.g., whether a sample is usable or whether the sample should be discarded. Advantages of using CZT detectors for radiation detection include the ability to perform spatially targeted measurements provided by the collimated nature of the detected signal of the CZT.

### DESCRIPTION OF THE DRAWINGS

Various example aspects of the systems and methods will be described in detail, with reference to the following figures, wherein:

FIG. 1 shows a system for radiopharmaceutical preparation according to an aspect of the invention;

FIG. 2A is a schematic of an HPLC process according to an aspect of the invention;

FIG. 2B illustrates a conventional HPLC loop design;

FIG. 2C illustrates an HPLC loop design according to various aspects of the current invention;

FIG. 3 shows a side view of an HPLC module according to an aspect of the invention;

FIG. 4 shows a front view of an HPLC module according to an aspect of the invention;

FIG. 5 shows a side view of an HPLC module according to an aspect of the invention;

FIG. 6 shows a perspective view of an HPLC module according to an aspect of the invention;

FIG. 7 shows a perspective view of an HPLC module according to an aspect of the invention;

FIG. 8 shows a perspective view of an HPLC module according to an aspect of the invention;

FIG. 9 shows a side view of an HPLC module according to an aspect of the invention;

FIG. 10 shows a side view of an HPLC module according to an aspect of the invention;

FIG. 11 shows a side view of an HPLC module according to an aspect of the invention;

FIG. 12 shows a front view of an HPLC module according to an aspect of the invention;

FIG. 13 shows a back view of an HPLC module according to an aspect of the invention;

FIG. 14 shows a perspective view of a multi-synthesis unit according to an aspect of the invention;

FIG. 15 shows a perspective view of a multi-synthesis unit according to an aspect of the invention;

FIG. 16 is a conceptual illustration of a gamma ray collimated detector in accordance with aspects of the disclosure;

FIG. 17 is a conceptual side illustration of the detector of FIG. 16;

FIG. 18 presents an example system diagram of various hardware components and other features, for use in accordance with an aspect of the present invention; and

FIG. 19 is a block diagram of various example system components, in accordance with an aspect of the present invention.

### DETAILED DESCRIPTION OF THE INVENTION

These and other features and advantages of this invention are described in, or are apparent from, the following detailed description of various example aspects.

Various aspects of a system for radiopharmaceutical preparation including a high performance liquid chromatography module may be illustrated by describing components that are coupled, attached, and/or joined together. As used herein, the terms “coupled”, “attached”, and/or “joined” are interchangeably used to indicate either a direct connection between two components or, where appropriate, an indirect connection to one another through intervening or intermediate components. In contrast, when a component is referred to as being “directly coupled,” “directly attached,” and/or “directly joined” to another component, there are no intervening elements shown in said examples.

Relative terms such as “lower” or “bottom” and “upper” or “top” may be used herein to describe one element's relationship to another element illustrated in the drawings. It will be understood that relative terms are intended to encompass different orientations of a system for radiopharmaceutical preparation in addition to the orientation depicted in the drawings. By way of example, if aspects of a system for radiopharmaceutical preparation shown in the drawings are turned over, elements described as being on the “bottom” side of the other elements would then be oriented on the “top” side of the other elements as shown in the relevant drawing. The term “bottom” can therefore encompass both an orientation of “bottom” and “top” depending on the particular orientation of the drawing.

Various aspects of a system for radiopharmaceutical preparation may be illustrated with reference to one or more examples of implementations. As used herein, the term “example” means “serving as an instance or illustration,” and should not necessarily be construed as preferred or advantageous over other variations of the devices, systems, or methods disclosed herein.

Aspects of the present invention relate to a system for radiopharmaceutical preparation involving solid and liquid phase interactions. The system may include a module for facilitating solid and liquid phase interactions by performing techniques including, but not limited to, high pressure, low pressure, and solid phase extraction. For example, the system may include a module for performing techniques including, but not limited to, HPLC, low pressure chromatography, flash chromatography, Sep-Pak® purification, and isolation processes. The system may include various modular components, each of which performs steps in the process of preparing radiopharmaceuticals. The modules may be added to and removed from the system easily to allow for flexibility in the operation of the system. According to an aspect of the invention, an HPLC module may be included to purify radiopharmaceuticals.

According to an aspect of the invention, modular components of a system for preparing radiopharmaceutical formulations may be placed in one or more containers. The container may be shielded to significantly reduce the amount of radiation leaving the container. The shielding material, which may include lead, may be joined to any or all sides of the container. In an aspect of the invention, a container may include one or more compartments for the placement of various modules.

Modules may be placed directly into the compartment or they may be placed within one or more frameworks that fit into the compartment and allows the modules to be added to and removed from the system easily. The framework may be of any size and may house any number of modules that may fit within the framework. The modules may include, but are not limited to, synthesis modules in which the radiopharmaceuticals are prepared and HPLC modules in which the radiopharmaceuticals are purified.

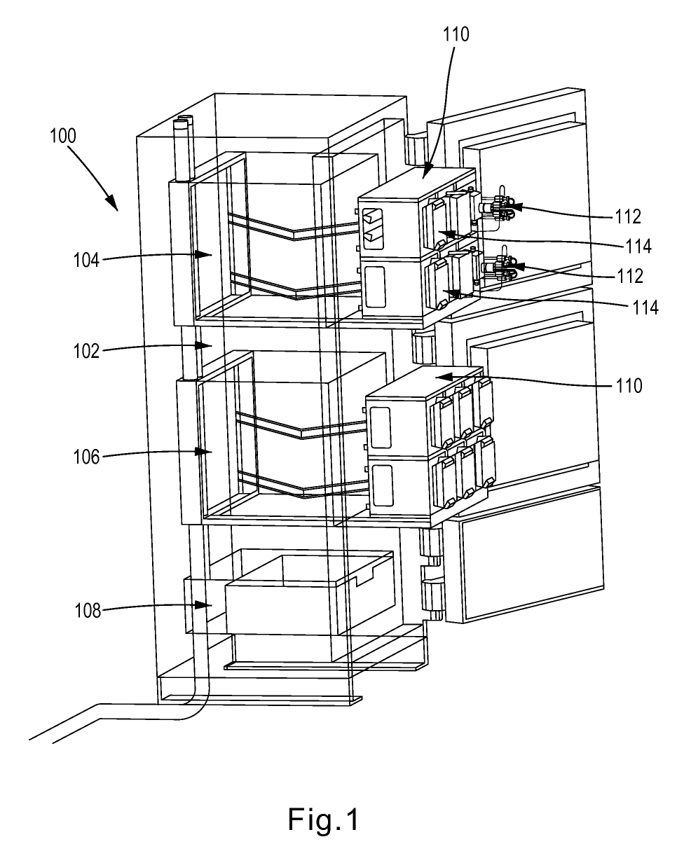

An example system according to an aspect of the invention is shown in FIG. 1. The system 100 may include a shielded container, such as a mini-cell 102. The mini-cell 102 may include compartments 104, 106, and 108. Compartments 104 and 106 may each house a framework for mounting specific units, such as multi-synthesis units 110. For example, a multi-synthesis unit 110 may be about 26-30 inches wide, 18-22 inches tall, and 18-24 inches deep. A multi-synthesis unit 110 may be placed on a sliding track, for example, within a compartment so that the multi-synthesis unit 110 can easily be accessed for service or replacement of the modules or their components.

Each multi-synthesis unit 110 may hold any number of modules as can be accommodated, while maintaining the overall compactness of the system 100. In the example shown in FIG. 1, the multi-synthesis units 110 may each incorporate up to six modules. For example, the modules may include, but are not limited to, HPLC modules 112 and synthesis modules 114. The components of the HPLC or synthesis modules 112 or 114 may be housed in a container that allows the modules to be removed easily from a multi-synthesis unit 110. A radiopharmaceutical material may be prepared in a synthesis module 114 and may then be purified in an HPLC module 112. One or more of the six modules in a mini-cell may be an HPLC module. An additional compartment 108 may be used to store waste products of the radiopharmaceuticals synthesis process of the system 100, for example.

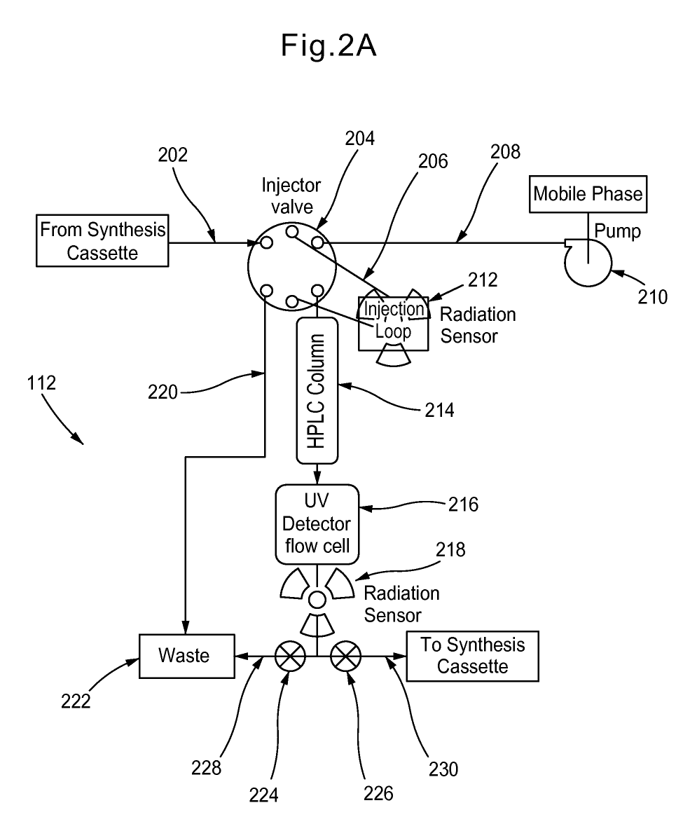

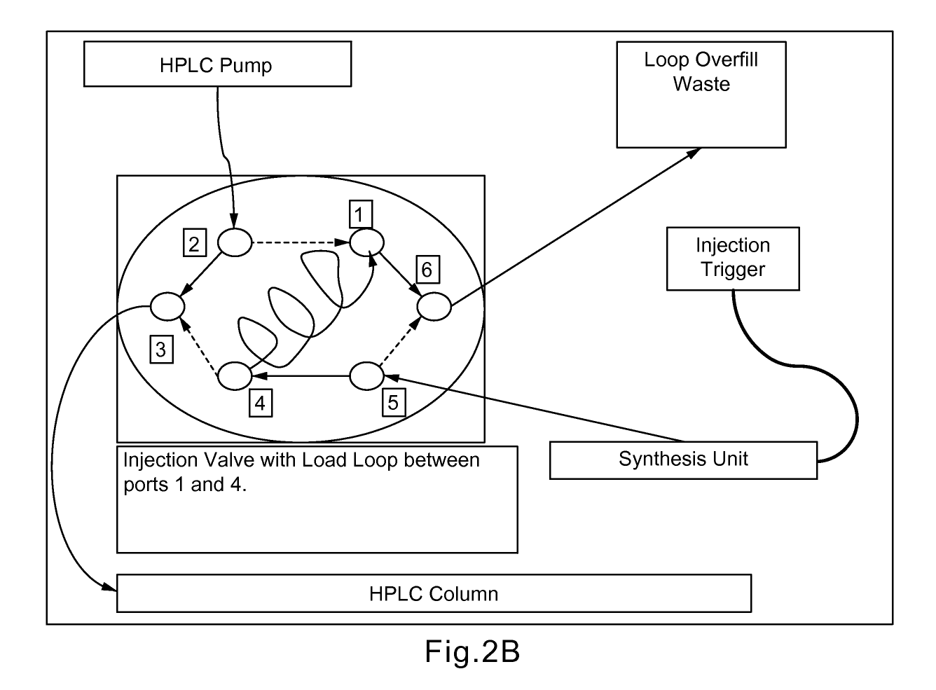

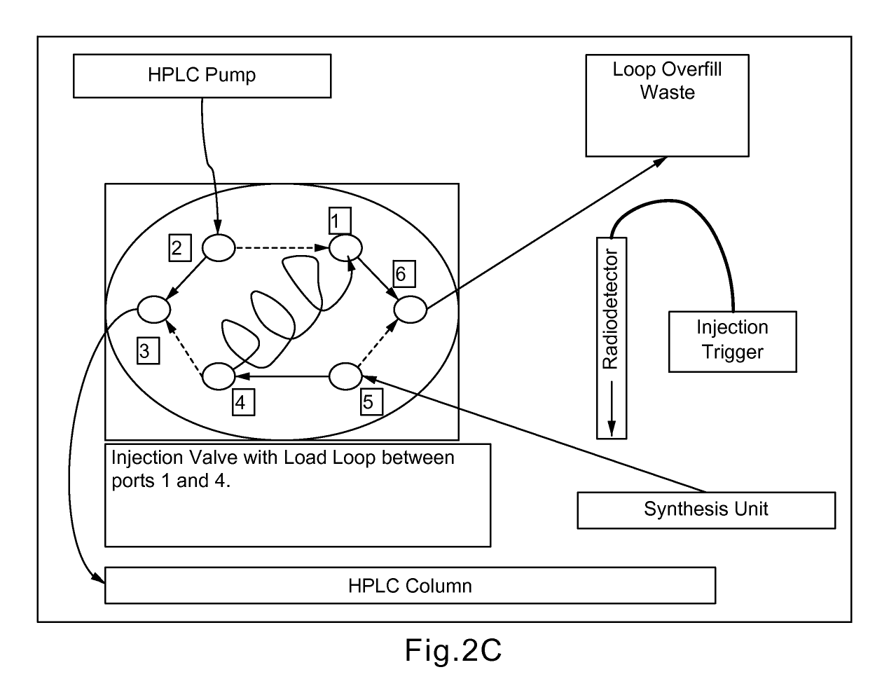

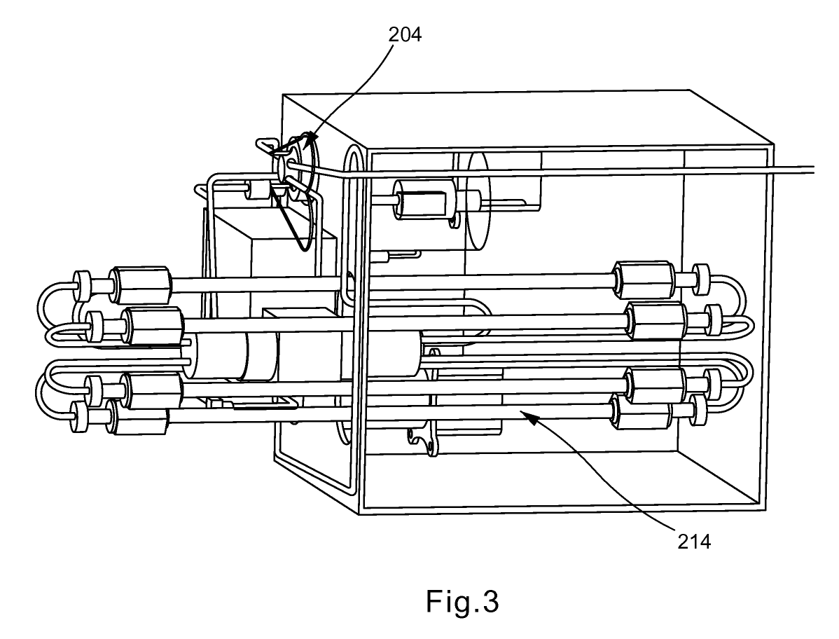

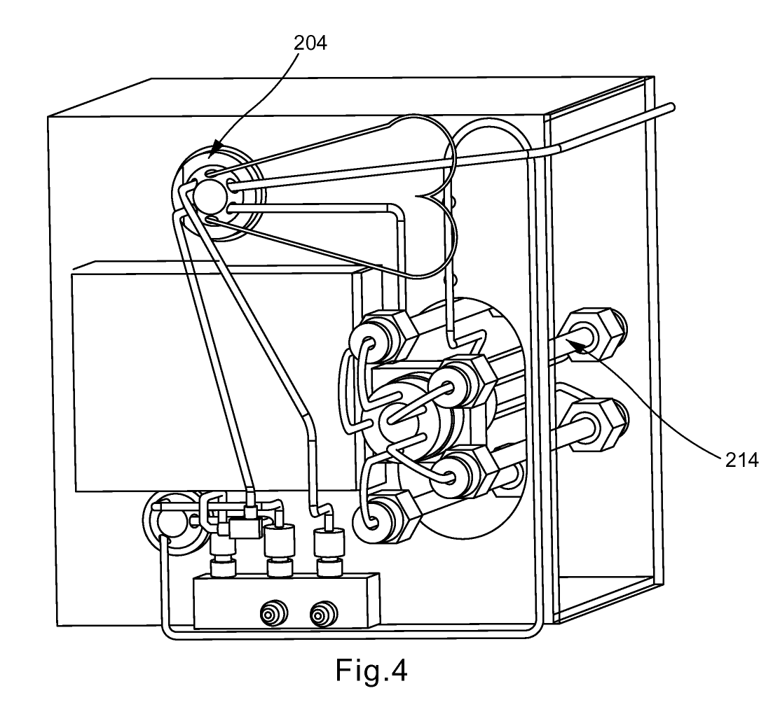

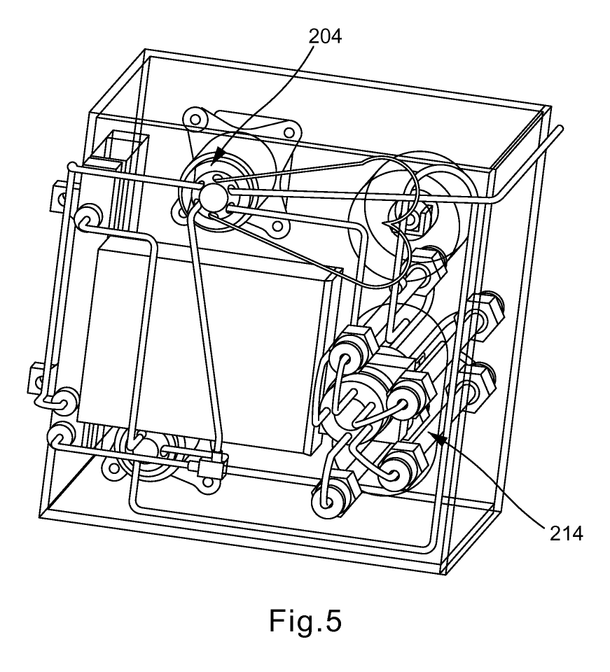

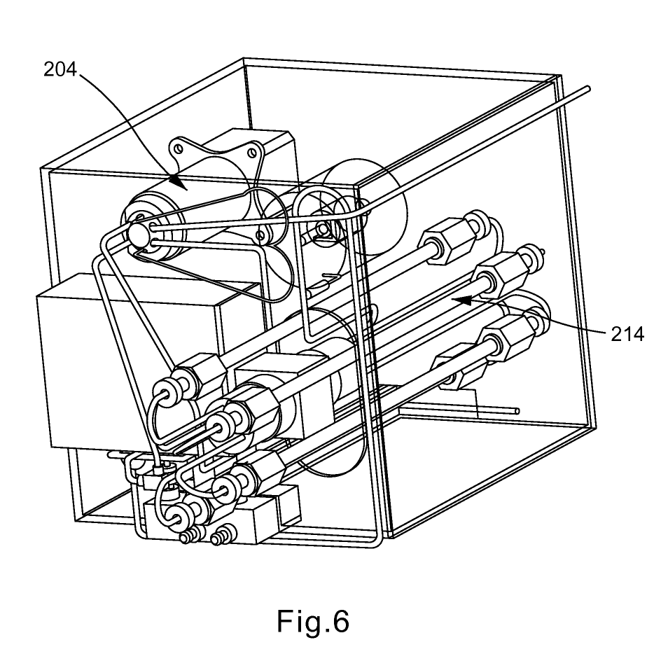

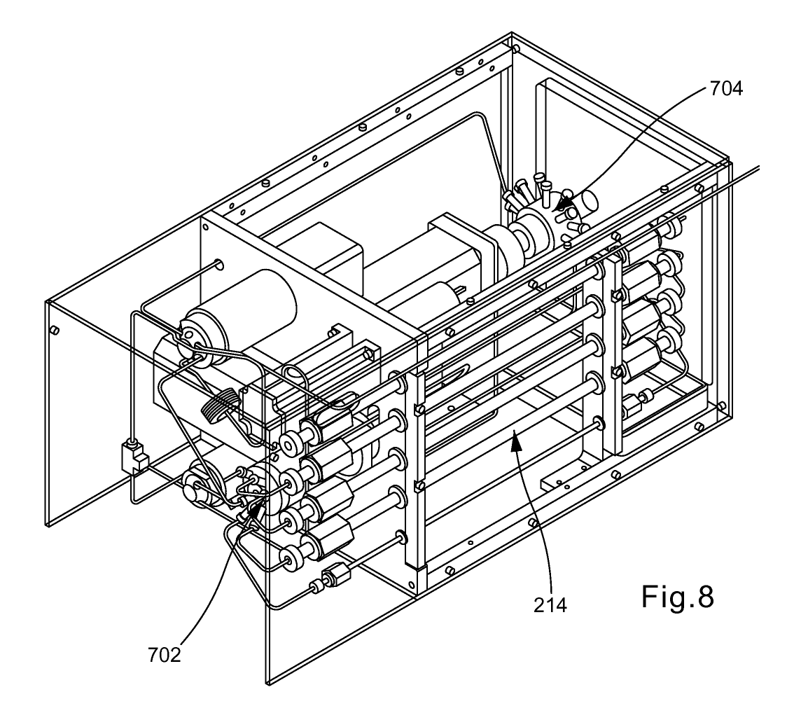

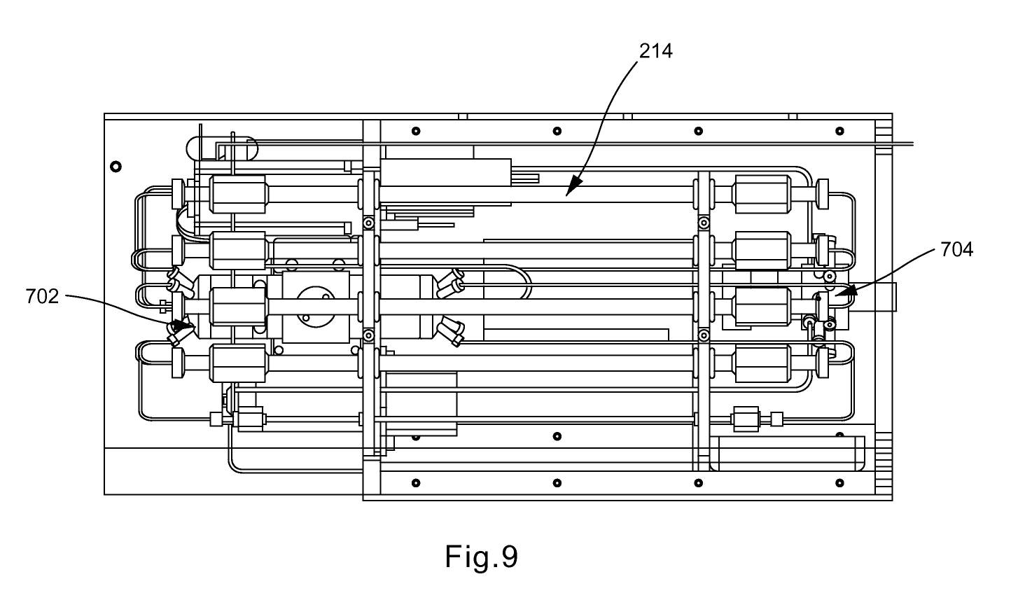

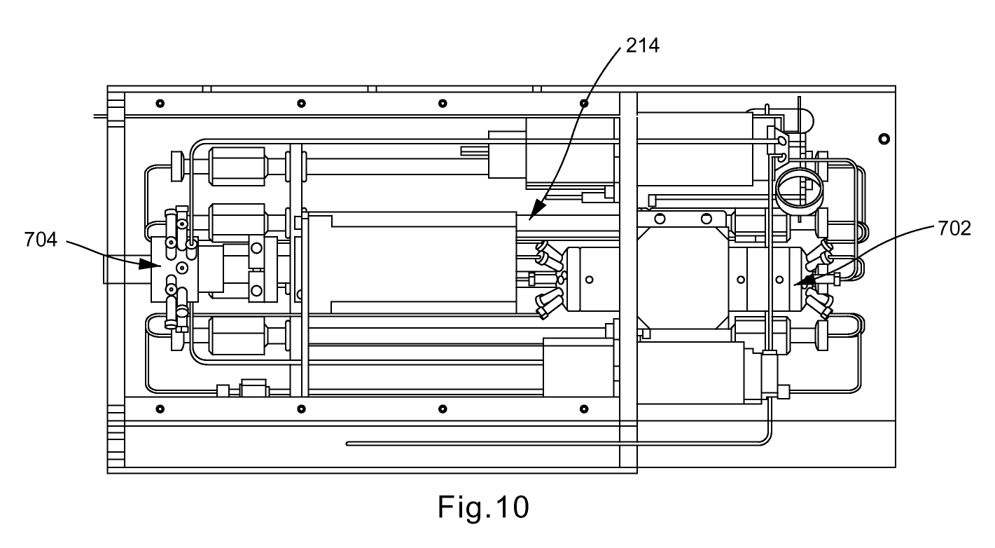

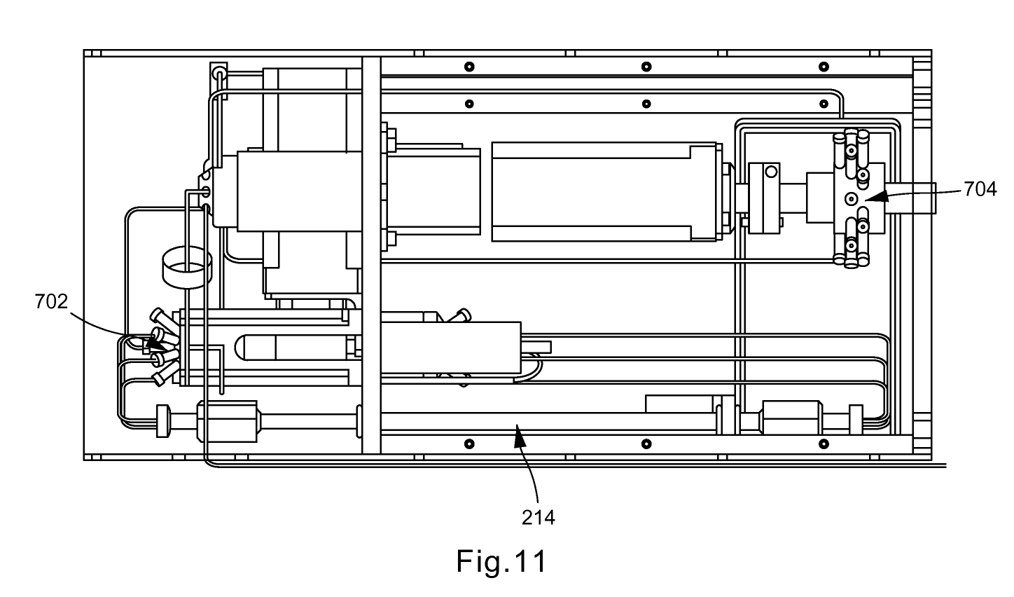

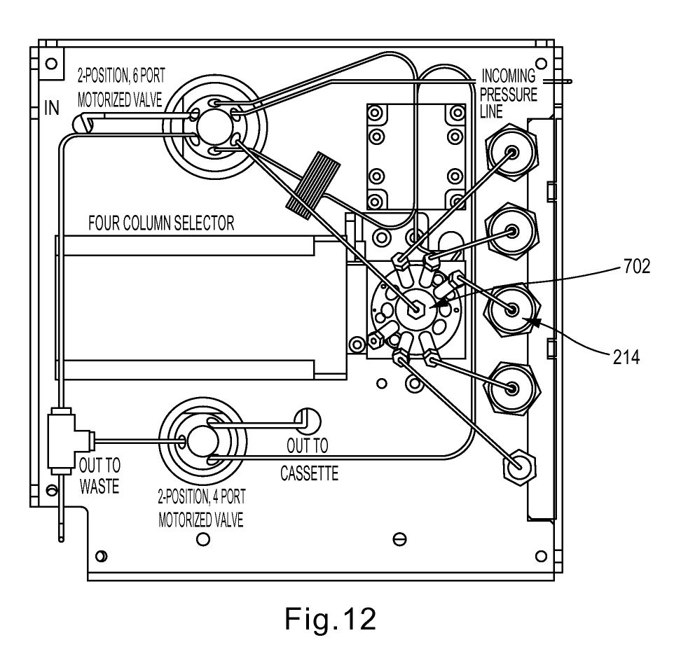

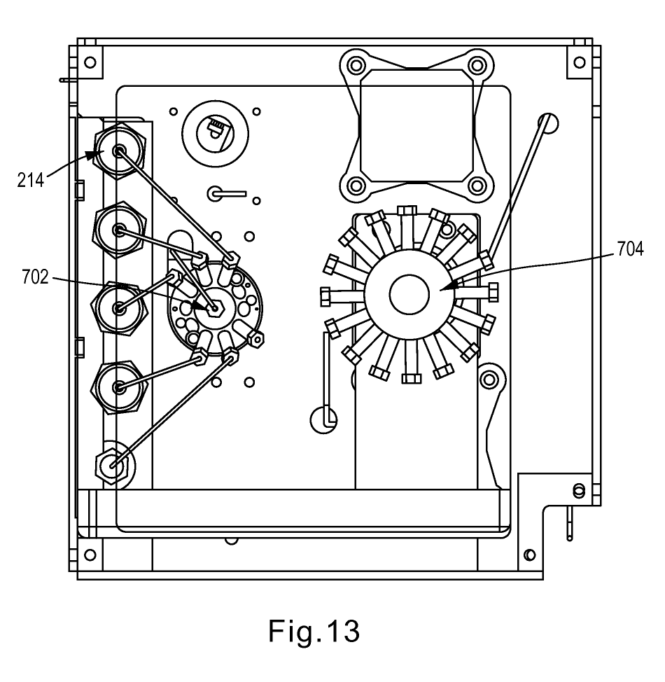

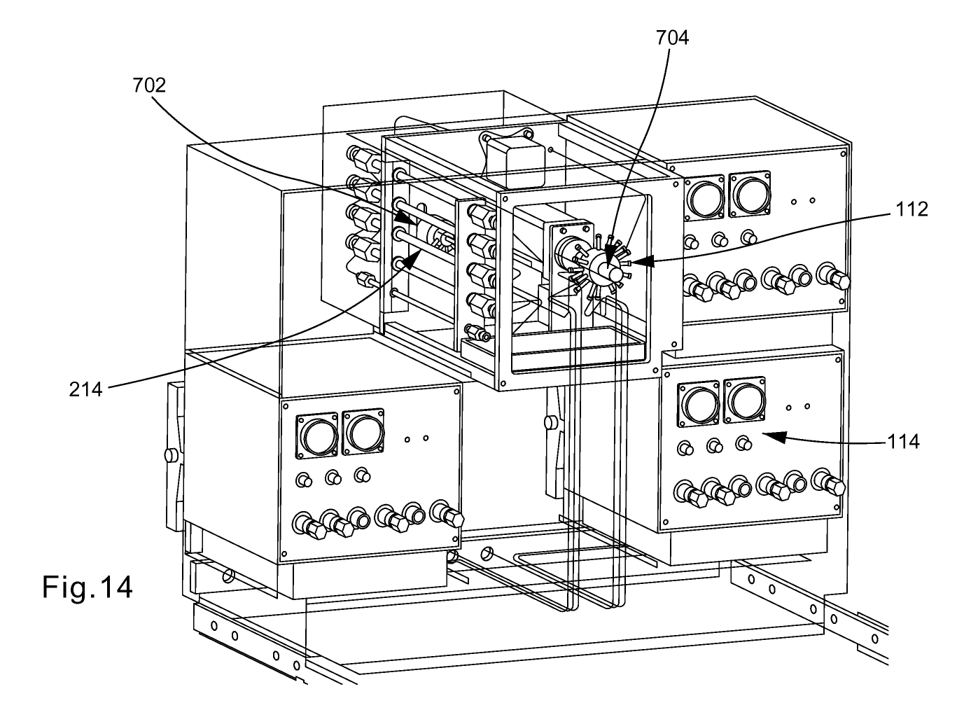

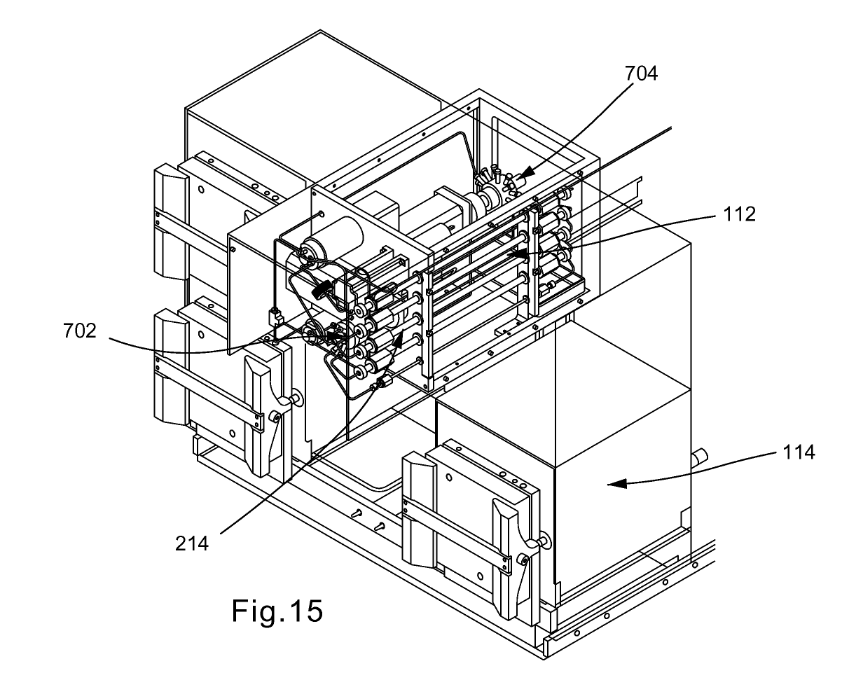

An HPLC module may include various components, including, but not limited to, one or more injector valves, injection loops, mobile phase solvent sources, high pressure pumps, columns, selector valves, sensors, and control components. The operation of an HPLC module according to an aspect of the invention is shown in FIG. 2A. Aspects of the HPLC module according to the present invention are shown in FIGS. 3-15, and like components are labeled with like reference numerals.

As shown in FIG. 2A, in the operation of an HPLC module 112, a radiopharmaceutical product that has been synthesized in a synthesis module 114 may move from an inlet 202 from the synthesis module and enter an injector valve 204. The radiopharmaceutical product may move from the injector valve 204 to an injection loop 206. The injection loop 206 may collect a quantity of the radiopharmaceutical product and inject it into a column 214. For example, 2 mL of radiopharmaceutical product may be held in the injection loop 206. The radiopharmaceutical product may stay in the injection loop 206 until separation in the column 214 is ready to begin. A radiation sensor 212 may be associated with the injection loop 206 to detect the presence of the radiopharmaceutical product and to determine that the entirety, or substantially the entirety, of the sample has been transferred from the injector valve 204. According to various aspects, the radiation sensor 212 may include a CZT sensor capable of detecting radiation emanating from the radiopharmaceutical product. As such, the radiation sensor 212 may be used to monitor the injection loop 206 by determining whether a product exiting from the injection loop 206 has a peak of radiation activity, indicating the presence of a radiopharmaceutical product. According to various aspects, there may also be one or more detectors such as, e.g., CZT detectors, at the waste line 220 to determine, for example, that no radiopharmaceutical product has been accidentally transferred to the waste line 220 to be eliminated as waste.

A mobile phase solvent, which may be held in a source, may be passed from the source to a mobile phase pump 210 where the mobile phase solvent may be pressurized. For example, any common mobile phase solvent, including, but not limited to, solutions of acetonitrile, isopropanol, and ethyl acetate, may be used. The mobile phase pump 210 may be a high pressure pump that may deliver a mobile phase and radiopharmaceutical product through a column 214. The pump may provide a pressure of about 500 to about 5000 psi, and preferably about 1,500 to about 3,000 psi. The pump may be set to provide the mobile phase at a specific flow rate. For example, the pump may provide the mobile phase at a rate of 5 mL/minute.

According to various aspects, although a single sensor 212 is depicted in FIG. 2A, a plurality of sensors 212 such as, e.g., CZT detectors, may also be used in different points of the injection loop 206 and/or the HPLC column 214 in order to follow the radiopharmaceutical product as the product travels through the loop 206 and the HPLC column 214. The path of the radiopharmaceutical product may be determined by detecting the movement of a peak of radioactivity. According to various aspects, the system may also include low pressure columns or disposable columns in addition to, or in place of, the HPLC column 214.

The mobile phase source and pump 210 may be positioned inside or outside the HPLC module 112. For example, the mobile phase source and pump 210 may be positioned outside of the multi-synthesis unit 110 or outside of the mini-cell 102. Positioning the mobile phase source and pump 210 outside of the HPLC module may reduce the overall size of the HPLC module. In such a configuration, the appropriate connections between the mobile phase source and pump 210 and the HPLC module may be made while maintaining the shielding of the mini-cell 102.

The pump 210 may be connectable to multiple mobile phase sources to allow the use of more than one mobile phase with an HPLC module 112. For example, the pump 210 may allow connections to four different mobile phase sources. In addition, the pump 210 may allow connections to additional fluids for use in flushing the system.

The mobile phase may move from the pump 210 through a mobile phase inlet 208 to the injector valve 204. When initiating the operation of the HPLC module 112, the mobile phase solvent may move from the injector valve 204 through a waste line 220 and flow to a waste tank 222. For example, this may be done to flush the injector valve 204 to remove any contaminating matter or residual fluids from a previous run of the HPLC module.

The mobile phase solvent may move from the injector valve 204 to column 214. Column 214 may be tightly packed with a stationary phase composition. The mobile phase may be passed to column 214 to “wet” or to condition the column 214 prior to beginning purification of the radiopharmaceutical product.

According to an aspect of the invention, once purification is set to begin, the radiopharmaceutical product may move from the injection loop 206, where it was collected, back to the injector valve 204. The injector valve 204, which is connected to the injection loop 206 and the mobile phase inlet 208, may operate by rotating to alternatively open the injection loop 206 and the mobile phase inlet 208. For example, the injector valve 204 may rotate to allow the radiopharmaceutical product held in the injection loop 206 to flow to the column, while disconnecting from the mobile phase inlet 208. Once the radiopharmaceutical product has entered the column, the injector valve 204 may rotate to allow the mobile phase to flow through the column, while disconnecting from the injection loop 206.

In column 214, the components of the radiopharmaceutical product may be separated based on their relative mobilities in the stationary phase contained in the column 214. According to an aspect of the invention, a particular column may be selected to contain the appropriate stationary phase material for the separation of the radiopharmaceutical product.

In general, any type or size of column that is typically used in HPLC or low pressure applications may be used in conjunction with aspects of the present invention. The stationary phase may include, but is not limited to, silica-based materials. The stationary phase may be for example, particles in granular form. The length and/or diameter of the columns used for some formulations may be reduced if fluid volumes and/or separation needs are reduced.

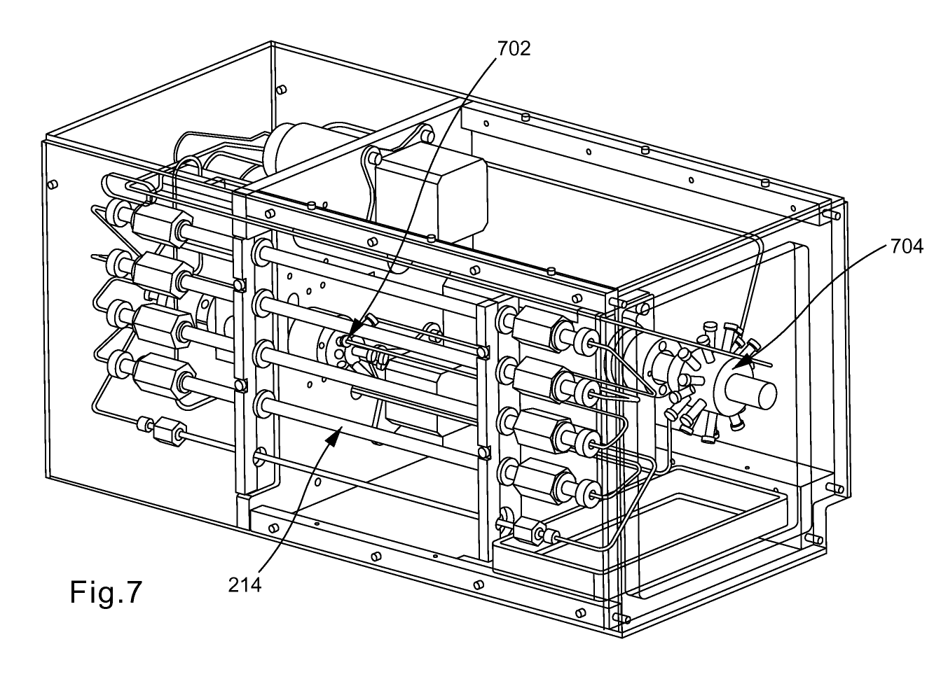

According to an aspect of the invention, more than one column 214 may be contained in an HPLC module 112 to provide increased flexibility in use of the overall system 100. For example, each column 214 in an HPLC module 112 may include a different stationary phase that may be used to purify a different radiopharmaceutical. The columns 214 may each be joined to a column selector valve 702, as shown in FIGS. 7-15, that allows a particular column to be selected for use in a run of the HPLC module 112. An HPLC module with multiple columns may also include a pump that may select from multiple solvent sources, as discussed above. This method provides increased flexibility in that it allows a wider range of radiopharmaceuticals to be made using a single HPLC module, reduces equipment costs, and reduces the amount of component and/or module swapping needed to accommodate a variety of radiopharmaceutical formulations.

FIG. 2B illustrates a conventional HPLC loop design. The semi-preparative HPLC loop load system uses a 2 position/6 port valve to transfer crude reaction mixture from the synthesis unit to the semi-preparative HPLC. The void volume between each of the ports is minimal. The synthesis unit uses a syringe driver to push approximately 4 mL of radioactive crude reaction mixture into the semi-preparative injection load loop. The mixture travels though approximately 18″ length of 0.03″ ID PEEK tubing prior to reaching port 5 of the injection valve. The mixture leaves port 4 and through port 5 enters the injection load loop, made of 0.03″ ID PEEK or Stainless Steel and containing approximately 5 mL volume. Any loop overflow enters port 1 and exists through port 6 to a loop overfill waste container. The mobile phase from the HPLC pump enters port 2 and exits from port 3 to the HPLC purification column. The current loading step is based on a pre-set timing before triggering the loop injection. Once the pre-set time is reached, the synthesis unit sends a 24V output to close a relay switch completing a circuit in the semi-preparative HPLC system. The resulting voltage drop triggers the connectivity between the valve ports to change (dashed lines). The new connection between ports 2 and 1 allows the mobile phase to enter the injection load loop, pushing the crude reaction mixture into the purification column via ports 4 and 3. However, an inherent problem with this system is the pre-set timings of the trigger initiation. The synthesis unit uses gas overpressure in the syringe to transfer the mixture to the load loop. Accordingly, drift over time in the overpressure can change the rate of loop loading, causing it to no longer meet the pre-set timing. The injection trigger generally occurs either too early or too late resulting in large losses of crude reaction mixture.

FIG. 2C illustrates an HPLC loop design according to various aspects of the current invention. According to various aspects, the same 2 position/6 port injection valve are similar to the ones discussed with respect to FIG. 2A. However a highly collimated, well shielded CZT radiodetector may be located along the crude reaction mixture transfer line between the synthesis unit and port 5 of the injection valve. The CZT radiodetector would be able to detect the transfer of radioactive material through this line, allowing the transfer to be monitored. Once the detection signal returns to a baseline, indicating complete transfer of the crude reaction mixture, the radio-detection software may be able to send a signal to the injection trigger to initiate injection on to the HPLC. Using this design, the synthesis unit would no longer be involved with triggering the HPLC injection. According to various aspects of the current invention, the activity range of the CZT detector may be between 100 mCi and 5000 mCi, the CZT detector may be capable of detecting reaction mixture from background noise, the size of the CZT detector may have an approximately 2″×2″×1″ footprint in minicell, and should be no larger than the current in off-the-shelf fluid detectors. According to various aspects, the CZT detector may be able to determine that a transfer is complete when the sensor returns to the baseline, and ignore “false positives” due to air bubbles in transfer mixture. As an output, a relay switch may be closed to initiate the injection trigger upon complete transfer of liquid. In addition, the transfer flow rate may be between 4 ml/min and 1 mL/min, due to gas overpressure specific to the synthesis unit. The flow rate may also decrease as transfer progresses due to gas overpressure equilibration.

For example, the HPLC module 112 may incorporate a four-way column selector valve 702 and four columns 214, as shown in FIGS. 3-15. Each of these columns may contain a different packing material, or some columns may contain the same type of material so that they may be used as backups for the other columns. The injection loop including the radiation detector 212 may be placed in the flow path ahead of the four-way column selector. As different formulations may require different solvents, a mobile phase pump that can select from four different solvent sources may be used. The pump may also have provisions to flush line sets in between applications. If columns of different lengths and diameter are used in the same HPLC module 112, they may be used with the same selector valve by making minor connection line changes. An example of a four-way column selector that may be used in the invention may be a six-way column to provide for flushing or cleaning paths.

One or more detectors may be positioned to detect the presence of the desired radiopharmaceutical material after the radiopharmaceutical product passes through the column 214. For example, the fluid exiting the column 214, which includes mobile phase and the components of the radiopharmaceutical product, may pass by a UV sensor 216 and/or a radiation sensor 218 or one or more CZT detectors. The UV sensor may alert the system that the constituents of interest are leaving the column. The radiation sensor 218 may detect the presence of the desired radiopharmaceutical material in the fluid exiting the column 214 and determine when the selected radioactive material is exiting the column. In another aspect of the invention, the HPLC module may include only one radiation sensor that may be used to detect the presence of radioactive compounds initially in the injection loop and later in the line downstream from the column. The use of only one sensor is advantageous in that it simplifies the instrumentation and control systems that are required for effective operation of the HPLC module of the invention.

According to various aspects, the radiation sensor 218 may include a CZT sensor capable of detecting radiation emanating from the radiopharmaceutical product. Accordingly, a measurement of the radioactivity level of a product exiting the HPLC column 214 can be achieved and a correct determination of whether to route the product to the waste line 228 or to the synthesis line 230 can be made based on the detected radioactivity level.

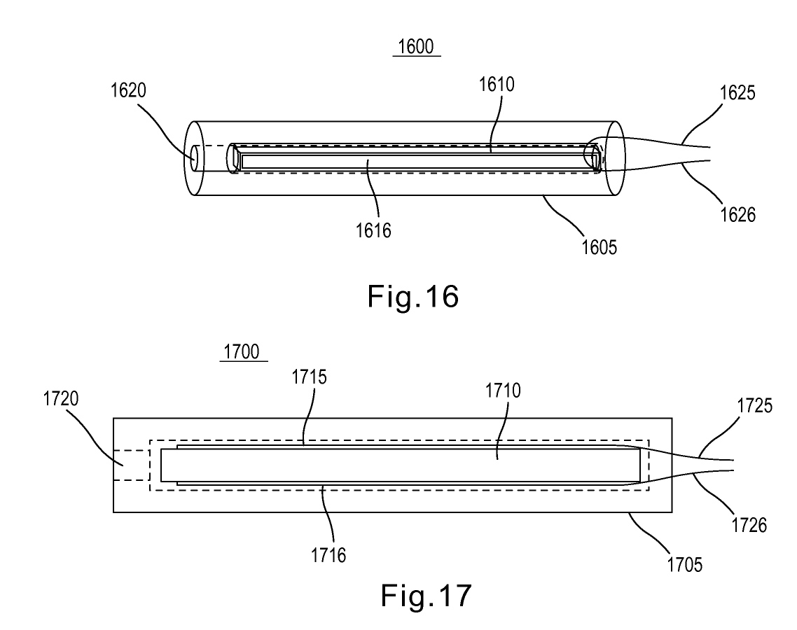

FIG. 16 shows a schematic illustration of a gamma ray collimated detector 1600. The sensor 1600 may include a Cadmium Zinc Telluride (CdZnTe, or CZT) element 1610, however, other solid state materials currently available or yet to be discovered may be used. CZT is a direct bandgap semiconductor and can operate in a direct-conversion (e.g., photoconductive) mode at room temperature, unlike some other materials (e.g., germanium) which may require cooling, in some cases, to liquid nitrogen temperature. The relative advantages of CZT over Germanium or other detectors include a high sensitivity for x-rays and gamma-rays, due to the high atomic numbers and masses of Cd and Te relative to other detector materials currently in use, and better energy resolution than scintillator detectors. A gamma ray (photon) traversing a CZT element 1610 liberates electron-hole pairs in its path. A bias voltage applied across electrodes 1615 (not shown in FIG. 16) and 1616 on the surface of the element 1610 (both shown in a side view in FIG. 17) causes a charge to be swept to the electrodes 1615, 1616 on the surface of the CZT (electrons toward an anode, holes toward a cathode). Wires 1625 and 1626 connect, respectively, from electrodes 1615 and 1616 to a source of the applied voltage.

The sensor 1600 can function accurately as a spectroscopic gamma energy sensor, particularly when element 1610 is CZT. However, geometric aspects may be considered. In conventional use of CZT as a gamma ray detector, the CZT element 1610 may be a thin platelet, which may be arranged in multiples to form arrays for imaging, generally perpendicularly facing the source of gamma ray emission. Therefore, gamma rays of differing energies traverse a detector element of substantially the same thickness. While absorption of the gamma ray may generally be less than 100% efficient, higher energy gamma rays will liberate more electron-hole pairs than lower energy gamma rays, producing a pulse of greater height. The spectrum and intensity of gamma ray energies may thus be spectroscopically determined by counting the number of pulses generated corresponding to different pulse heights.

Because higher energy photons may travel a greater distance in the CZT rod 1610 before complete absorption, it is advantageous for the CZT rod 1610 to be longer in a longitudinal direction (i.e., along a long axis) intersecting a known source volume of radionuclide being measured. Gamma rays incident on the CZT rod off of, or transverse to, the long axis may not be fully absorbed, and thus, the CZT rod will not be as sensitive a detector of such gamma rays as a result. Thus, elongating the CZT rod in one direction introduces a degree of collimation and directional sensitivity along the extended direction.

The absorption coefficient for 511 keV gamma ray absorption in CZT is μ=0.0153 cm²/gm. The absorption probability as a function of μ, density ρ(=5.78 gm/cm³) and penetration distance h is

P(μ,h)=1−e^{-μρh}

Therefore, the ratio of absorption in a 10 mm length of CZT to a 1 mm length is

$$\frac{P ( μ , 10 mm )}{P ( μ 1 mm )} ∼ 9.613 .$$

That is, the directional sensitivity for gamma ray detection of CZT at 511 keV along the 10 mm length of the detector is nearly 10 times greater than in the 1 mm thick transverse direction.

Referring to FIG. 17, the sensor may be a CZT rod 1710 as just described, encased in a shielded case 1705 (e.g., tungsten) with an aperture 1720 open and directed toward the vial containing radiopharmaceutical to expose the CZT rod 1710 along the long dimension of the rod 1710, while shielding the CZT rod 1710 from gamma rays incident laterally to the long dimension of the rod 1710, e.g., from directions other than along the long dimension. Therefore, the combination of shielding, aperture and extended length of the CZT detector in direction of gamma ray emission from a portion of the radiopharmaceutical sample provides a substantial directional “virtual” collimation of the CZT detector's sensitivity to gamma rays incident from the container in a volume of radionuclide defined by the collimation and the size (e.g., diameter) of the container and the collimation of the acceptance aperture 1720 of the detector 1700. Because the volume of the radiopharmaceutical “observable” by the sensor is constant from measurement to measurement, the concentration and activity can be determined after calibration.

Based on the data from UV sensor 216 and/or a radiation sensor 218, selector valves 224 and 226, or a single 3 way valve (positioned between 224 and 226) may be actuated to direct the fluid exiting the column 214 to the appropriate outlets. For example, when the radiation sensor 218 detects the presence of the desired radiopharmaceutical material in the fluid flowing out of the column 214, selector valve 226 may be opened to allow the radiopharmaceutical material to move through a return line from the selector valve 226 to the synthesis module 114. When the radiation sensor 218 does not detect the presence of the desired radiopharmaceutical material in the fluid flowing out of the column 214, selector valve 224 may be opened to allow the mobile phase and other components from the radiopharmaceutical product to move through a line from the selector valve 224 to the waste container 222.

According to aspects of the present invention, the desired radiopharmaceutical material moves back to the synthesis module 114 from which the radiopharmaceutical product that was processed through the HPLC module came. Alternatively, the desired radiopharmaceutical material may move to a different module, including, but not limited to another synthesis module or another HPLC module.

The operation of an HPLC module, according to aspects of the present invention, may be aided by various control components and sensor instrumentation. For example, the actuation of components including the injector valve 204, pump 210, column selector, and selector valves 224 and 226 may be done by control components that may be positioned within or external to an HPLC module 112. The control components may respond to inputs from a user or computer who specifies which radiopharmaceutical they would like to prepare.

Components of the HPLC module of aspects of the present invention, including, but not limited to the injector valve, injector loop, columns, and sensors, may be reused after flushing. The HPLC module of aspects of the present invention may also include any number of additional sources of fluids and tubing to aid in cleaning and flushing the system. Any waste materials may be directed to a waste container.

Various components of the HPLC module of aspects of the present invention may be also replaced periodically, or be changed when used for synthesizing different radiopharmaceutical compounds. Typically, a component of the system may be replaced while the system is not operating to prepare a radiopharmaceutical. For example, after a column may be used in multiple runs of the HPLC module to purify radiopharmaceutical products, it may deteriorate or not function effectively to separate the desired radiopharmaceutical material. Failure of a column is usually detected, for example, by pressure changes either higher or lower than normal in the pump at a set mobile phase flow rate or changes in the processing time for separating the desired radiopharmaceutical material than is typically required. Such a failure may occur after about 10 to 100 runs of the HPLC module with that column. According to aspects of the present invention, the deteriorated column may be replaced with a different column.

In addition, a column having a specific stationary phase may be required to produce a particular radiopharmaceutical compound. Thus, according to aspects of the present invention, a column in an HPLC module having one stationary phase may be removed from the HPLC module and replaced with another column that has a different stationary phase. To simplify the removal of a column from the HPLC module, at least one side of the container that houses the HPLC module's components may be removed and columns may be positioned near that side of the HPLC module, as shown in FIGS. 7-8, and 12-15.

According to an aspect of the present invention, an HPLC module may be installed adjacent to a synthesis module in the same or a different multi-synthesis unit with fluid connections between the modules so that the radiopharmaceutical material in the synthesis module may be transferred to the HPLC module for purification. In one aspect of the present invention, one HPLC module may be installed for used with each synthesis module that requires HPLC processing. For example, in a multi-synthesis unit with spaces for six modules, three HPLC modules may be installed adjacent to three synthesis modules.

In another aspect of the present invention, a single HPLC module may be used with more than one synthesis module. For example, an HPLC module may allow inputs from more than one synthesis module using a selector valve 704 that allows a particular synthesis module to be selected for use in a run of the HPLC module 112, as shown in FIGS. 7-12, 14 and 15. While only one batch of radiopharmaceutical product may generally be processed by an HPLC module at a time, if one HPLC module may be used with several synthesis modules sequentially, the column selector valve 702 may be used to channel the flow of the radiopharmaceutical product and the appropriate mobile phase to the appropriate column in the HPLC module. Use of a single HPLC module with multi-synthesis modules may make more space available in a multi-synthesis unit and allow a single multi-synthesis unit to prepare more types of radiopharmaceuticals.

In general, according to the invention, modules in the same or different multi-synthesis units may be removably connected to one another by quick disconnects and hands-free connections to provide fluid communication of multiple ingredients and/or reagents between the modules. Various types of modules may be joined together so that significant manipulations are not required when adding, removing, or replacing modules. The fluid connection points between a synthesis module and an HPLC module may allow the modules to be positioned anywhere in the multi-synthesis units. For example, connections to a synthesis module may be made by connection points at the side of the module. As a synthesis cassette, which may be used with a synthesis module, is pulled into contact with the synthesis module, the cassette may also make connections to an HPLC module.

In addition, quick disconnects and hands-free connections between the modules and the multi-synthesis unit's back plane may be used to facilitate rapid module replacement. In addition, a cable management system may be used to permit an entire multi-synthesis unit to be pulled forward in the compartment for service without disconnecting fluid, gas, and electrical lines from the multi-synthesis unit.

Due to the high pressures in an HPLC column, components of the HPLC module of the invention that are connected in the high pressure portions of the circuit may be connected using threaded fittings. To simplify the process of removing and replacing components, the lines in the low pressure portions of the circuit, including the lines to and from the synthesis module, may be non-threaded.

According to aspects of the present invention, various approaches may be used to simplify the configuration of the HPLC module for preparing a specific radiopharmaceutical formulation. For example, a different HPLC module may be configured for each formulation, and they may each be easily installed and removed from a multi-synthesis unit. The system may also be configured with different mobile phase solvent sources and different columns in the HPLC modules, as discussed above. For example, the system may be configured so that a column with the appropriate stationary phase is in place for the next formulation that is scheduled to be prepared. In addition, the radiopharmaceutical formulations that may be used in accordance with the invention may be revised or adapted to use fewer column stationary phase materials and mobile phase solvents, thus simplifying the design of the HPLC module. This may require changes in the formulation recipes.

Due to space constraints and the radiation activity levels inside the mini-cell, the various components of the HPLC module of the invention may be located outside of the HPLC module, outside of the multi-synthesis unit that the HPLC module is housed in, or outside of the mini-cell. For example, the solvent pump, solvent supply systems, sensor electronics, and the control system for the selector valves may be located outside of the mini-cell. In addition, the HPLC module or some of the HPLC components may be located in a different multi-synthesis unit from a synthesis module to which it is connected. The placement of components outside of an HPLC module or outside of the mini-cell may be advantageous in that it may reduce the overall size of the system and provide easier access to those external components.

The system of aspects of the present invention may synthesize a variety of radiopharmaceutical formulations that require HPLC processing. The radiopharmaceuticals may include, but are not limited to, FLT, F-MISO, FBB, AV-45, AV-133, and F18. Different stationary phase column packing materials and mobile phase solvents may be used for synthesizing different radiopharmaceutical formulations, in accordance with FDA-approved standards.

According to aspects of the present invention, when an operator provides an input to the system, such as the selection of a particular radiopharmaceutical or a particular synthesis cassette and reagent pack for use with a synthesis module, the system may instruct the HPLC module to perform its function in that particular radiopharmaceutical production run. Further, the system may specify the combination of mobile phase and column that should be used in the HPLC process. Alternatively, the use of an HPLC module in a radiopharmaceutical production run and its specific mobile phase and column may be selected by an operator. In addition, if a certain radiopharmaceutical does not require HPLC processing, the system will not channel the radiopharmaceutical product from the synthesis module to an HPLC module.

An example method of preparation of a radiopharmaceutical using a system according to an aspect of the invention is described below.

The radiopharmaceutical Fluorothymidine F 18 ([¹⁸F]FLT) in injectable form may be purified using semi-prep HPLC. After hydrolysis and neutralization, 4 mL of a crude [¹⁸F]FLT product solution may be diluted with 1.0 mL of a mobile phase that includes 8% ethanol and 92% 10 mM phosphate buffer. The solution may then be transferred through an Alumina N Sep-Pak cartridge and into a sample loop on an injection valve. The contents of the sample loop (5 mL) may be injected onto an HPLC column and purified using the mobile phase. The mobile phase flow rate may be 5 mL/min, which generate a system pressure of about 15 MPa or 2000 psi. Under these conditions, the [¹⁸F]FLT product may be eluted from the column and may be collected at 16-18 minutes. The yield of [¹⁸F]FLT based on the starting [¹⁸F]fluoride ion may be approximately 20-25% (uncorrected for decay).

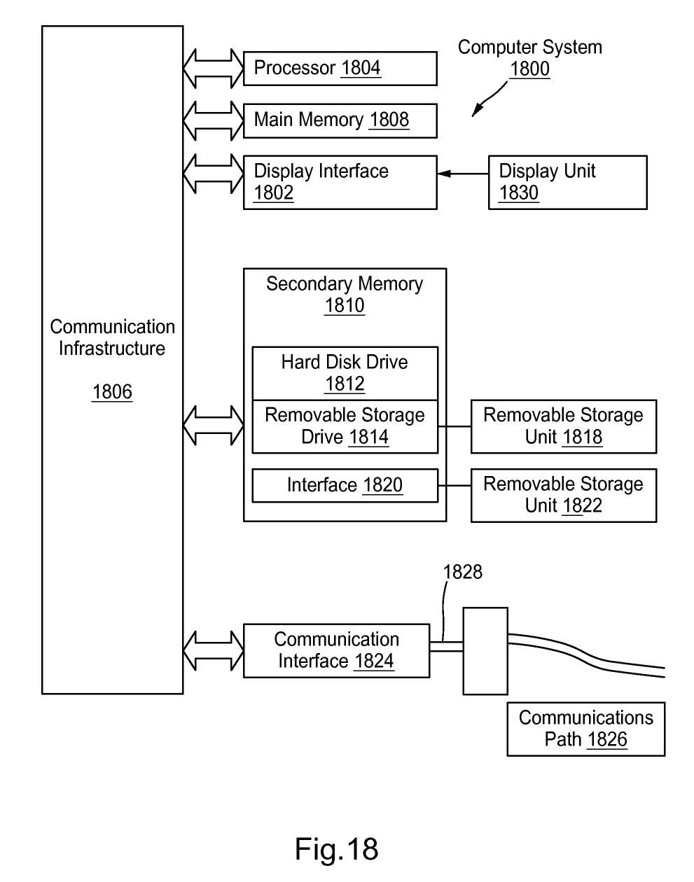

FIG. 18 presents an example system diagram of various hardware components and other features, for controlling the system in accordance with an aspect of the present invention. The present invention may be implemented using hardware, software, or a combination thereof and may be implemented in one or more computer systems or other processing systems. In one aspect, the invention is directed toward one or more computer systems capable of carrying out the functionality described herein. An example of such a computer system 1800 is shown in FIG. 18.

Computer system 1800 includes one or more processors, such as processor 1804. The processor 1804 is connected to a communication infrastructure 1806 (e.g., a communications bus, cross-over bar, or network). Various software aspects are described in terms of this example computer system. After reading this description, it will become apparent to a person skilled in the relevant art(s) how to implement the invention using other computer systems and/or architectures.

Computer system 1800 can include a display interface 1802 that forwards graphics, text, and other data from the communication infrastructure 1806 (or from a frame buffer not shown) for display on a display unit 1830. Computer system 1800 also includes a main memory 1808, preferably random access memory (RAM), and may also include a secondary memory 1810. The secondary memory 1810 may include, for example, a hard disk drive 1812 and/or a removable storage drive 1814, representing a floppy disk drive, a magnetic tape drive, an optical disk drive, etc. The removable storage drive 1814 reads from and/or writes to a removable storage unit 1818 in a well-known manner. Removable storage unit 1818, represents a floppy disk, magnetic tape, optical disk, etc., which is read by and written to removable storage drive 1814. As will be appreciated, the removable storage unit 1818 includes a computer usable storage medium having stored therein computer software and/or data.

In alternative aspects, secondary memory 1810 may include other similar devices for allowing computer programs or other instructions to be loaded into computer system 1800. Such devices may include, for example, a removable storage unit 1822 and an interface 1820. Examples of such may include a program cartridge and cartridge interface (such as that found in video game devices), a removable memory chip (such as an erasable programmable read only memory (EPROM), or programmable read only memory (PROM)) and associated socket, and other removable storage units 1822 and interfaces 1820, which allow software and data to be transferred from the removable storage unit 1822 to computer system 1800.

Computer system 1800 may also include a communications interface 1824. Communications interface 1824 allows software and data to be transferred between computer system 1800 and external devices. Examples of communications interface 1824 may include a modem, a network interface (such as an Ethernet card), a communications port, a Personal Computer Memory Card International Association (PCMCIA) slot and card, etc. Software and data transferred via communications interface 1824 are in the form of signals 1828, which may be electronic, electromagnetic, optical or other signals capable of being received by communications interface 1824. These signals 1828 are provided to communications interface 1824 via a communications path (e.g., channel) 1826. This path 1826 carries signals 1828 and may be implemented using wire or cable, fiber optics, a telephone line, a cellular link, a radio frequency (RF) link and/or other communications channels. In this document, the terms “computer program medium” and “computer usable medium” are used to refer generally to media such as a removable storage drive 1880, a hard disk installed in hard disk drive 1870, and signals 1828. These computer program products provide software to the computer system 1800. The invention is directed to such computer program products.

Computer programs (also referred to as computer control logic) are stored in main memory 1808 and/or secondary memory 1810. Computer programs may also be received via communications interface 1824. Such computer programs, when executed, enable the computer system 1800 to perform the features of the present invention, as discussed herein. In particular, the computer programs, when executed, enable the processor 1810 to perform the features of the present invention. Accordingly, such computer programs represent controllers of the computer system 1800.

In an aspect where the invention is implemented using software, the software may be stored in a computer program product and loaded into computer system 1800 using removable storage drive 1814, hard drive 1812, or communications interface 1820. The control logic (software), when executed by the processor 1804, causes the processor 1804 to perform the functions of the invention as described herein. In another aspect, the invention is implemented primarily in hardware using, for example, hardware components, such as application specific integrated circuits (ASICs). Implementation of the hardware state machine so as to perform the functions described herein will be apparent to persons skilled in the relevant art(s).

In yet another aspect, the invention is implemented using a combination of both hardware and software.

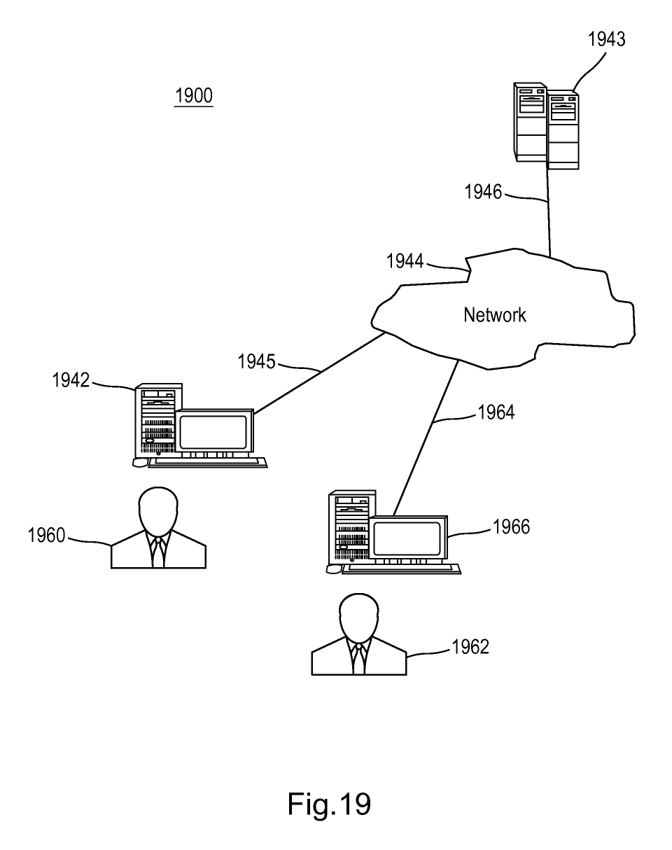

FIG. 19 is a block diagram of various example system components, in accordance with an aspect of the present invention. FIG. 19 shows a communication system 1900 usable in accordance with the present invention. The communication system 1900 includes one or more accessors 1960, 1962 (also referred to interchangeably herein as one or more “users”) and one or more terminals 1942, 1966. In one aspect, data for use in accordance with the present invention is, for example, input and/or accessed by accessors 1960, 1962 via terminals 1942, 1966, such as personal computers (PCs), minicomputers, mainframe computers, microcomputers, telephonic devices, or wireless devices, such as personal digital assistants (“PDAs”) or a hand-held wireless devices coupled to a server 1943, such as a PC, minicomputer, mainframe computer, microcomputer, or other device having a processor and a repository for data and/or connection to a repository for data, via, for example, a network 1944, such as the Internet or an intranet, and couplings 1945, 1946, 1964. The couplings 1945, 1946, 1964 include, for example, wired, wireless, or fiberoptic links. In another aspect, the method and system of the present invention operate in a stand-alone environment, such as on a single terminal.

While aspects of this invention have been described in conjunction with the example features outlined above, various alternatives, modifications, variations, improvements, and/or substantial equivalents, whether known or that are or may be presently unforeseen, may become apparent to those having at least ordinary skill in the art. Accordingly, the example aspects of the invention, as set forth above, are intended to be illustrative, not limiting. Various changes may be made without departing from the spirit and thereof. Therefore, aspects of the invention are intended to embrace all known or later-developed alternatives, modifications, variations, improvements, and/or substantial equivalents.

What is claimed is:

1. A system for radiopharmaceutical production, comprising:
a first synthesis module configured to synthesize a radiopharmaceutical product;
an injector valve capable of collecting a portion of the radiopharmaceutical product, the injector valve being coupled to an injector loop;
an analysis column configured to receive the portion of the radiopharmaceutical product collected from the injector valve via the injector loop; and
one or more detectors configured to detect one or more characteristics of the portion of the radiopharmaceutical product.

2. The system of claim 1, wherein the one or more detectors comprise at least one of an ultra-violet detector and a radiation sensor.

3. The system of claim 2, wherein the radiation sensor comprises at least one Cadmium Zinc Telluride (CZT) detector.

4. The system of claim 2, wherein the radiation sensor detects a radiation level of the portion of the radiopharmaceutical product and is located adjacent to at least one of the injector loop, an output of the analysis column, an output of the ultra-violet detector, one or more locations of the injector valve, and a waste path of the radiopharmaceutical product.

5. The system of claim 4, further comprising a second synthesis module, wherein the portion of the radiopharmaceutical product is transferred to the second synthesis module based on a radiation level detected by the one or more radiation detectors.

6. The system of claim 1, wherein the analysis column comprises at least one of a high performance liquid chromatography column, a low pressure chromatography column, a flash chromatography column, a purification module and an isolation module.

7. A method of radiopharmaceutical production, comprising:
transferring a radiopharmaceutical product from a first synthesis module to an injector valve, the injector valve being configured to inject one or more portions of the radiopharmaceutical product into an analysis column;
transferring at least one portion of the radiopharmaceutical product from the injector valve to the analysis column via an injection loop;
performing a reaction with the at least one portion of radiopharmaceutical product in the analysis column;
detecting at least one of an ultra-violet signal and a radiation level of the at least one portion of the radiopharmaceutical product; and
transferring the at least one portion of the radiopharmaceutical product to one of a second synthesis module and a waste container based on the detection.

8. The method of claim 7, wherein the detecting is of the radiation level, and the detecting further comprises providing one or more radiation sensors adjacent to at least one of the injector loop, an output of the analysis column, an output of the ultra-violet detector, one or more locations of the injector valve, and a waste path of the radiopharmaceutical product.

9. The method of claim 7, wherein the detecting is of the radiation level, and the detecting is performed at a time selected from a group consisting of: before the reaction is performed, during the reaction being performed, and after the reaction has been performed.

10. The method of claim 7, wherein performing the reaction further comprises performing at least one of a high performance liquid chromatography, a low pressure chromatography column, a flash chromatography, a solid-liquid separation and a purification.

11. The method of claim 7, wherein the detecting is of the radiation level and the detecting is performed via a CZT detector.

12. The method of claim 7, wherein:
the at least one portion of the radiopharmaceutical product is transferred to the second synthesis module when the detected radiation level is equal to or above a given threshold; and
the at least one portion of the radiopharmaceutical product is transferred to the waste container when the detected radiation level is below the given threshold.

13. The method of claim 7, wherein the detecting is of the ultra-violet signal, the detecting further comprising providing an ultra-violet detector at an output of the analysis column.

14. A computer program product comprising a computer usable medium having control logic stored therein for causing a computer to control radiopharmaceutical production, the control logic comprising:
computer readable program code means for controlling transferring a radiopharmaceutical product from a first synthesis module to an injector valve, the injector valve being configured to inject one or more portions of the radiopharmaceutical product into an analysis column;
computer readable program code means for controlling transferring at least one portion of the radiopharmaceutical product from the injector valve to the analysis column via an injection loop;
computer readable program code means for controlling performing a reaction with the at least one portion of radiopharmaceutical product in the analysis column;
computer readable program code means for detecting at least one of an ultra-violet signal and a radiation level of the at least one portion of the radiopharmaceutical product; and
computer readable program code means for controlling transferring the at least one portion of the radiopharmaceutical product to one of a second synthesis module and a waste container based on the detection.

15. A system for radiopharmaceutical production, the system comprising:
a processor;
a user interface functioning via the processor; and
a repository accessible by the processor; wherein
a radiopharmaceutical product is transferred from a first synthesis module to an injector valve, the injector valve being configured to inject one or more portions of the radiopharmaceutical product into an analysis column;
at least one portion of the radiopharmaceutical product is transferred from the injector valve to the analysis column via an injection loop;
a reaction with the at least one portion of radiopharmaceutical product is performed in the analysis column;
at least one of an ultra-violet signal and a radiation level of the at least one portion of the radiopharmaceutical product is detected; and
the at least one portion of the radiopharmaceutical product is transferred to one of a second synthesis module and a waste container based on the detection.

16. The system of claim 15, wherein the processor is housed on a terminal selected from a group consisting of a personal computer, a minicomputer, a main frame computer, a microcomputer, a hand held device, and a telephonic device.

17. The system of claim 15, wherein the processor is housed on a server selected from a group consisting of a personal computer, a minicomputer, a microcomputer, and a main frame computer.

18. The system of claim 17, wherein the server is coupled to a network via a coupling.

19. The system of claim 18, wherein the network is the Internet.

20. The system of claim 18, wherein the coupling is selected from a group consisting of a wired connection, a wireless connection, and a fiberoptic connection.
